(19) United States
(12) Patent Application Publication
(10) Pub. No.: US 2024/0382162 A1
(43) Pub. Date: Nov. 21, 2024

(54) Method for Providing Information of Major Depressive Disorders and Device for Providing Information of Major Depressive Disorders Using the Same

(71) Applicant: B-Wave Co., Ltd., Goyang-si Gyeonggi-do (KR)

(72) Inventors: Seung Hwan Lee, Goyang-si Gyeonggi-do (KR); Han Jeong Hwang, Jochiwon-eup Sejong (KR); Mi Seon Shim, Jochiwon-eup Sejong (KR)

(73) Assignee: B-Wave Co., Ltd., Goyang-si Gyeonggi-do (KR)

(21) Appl. No.: 18/693,521

(22) PCT Filed: Sep. 27, 2022
(86) PCT No.: PCT/KR2022/014471

§ 371 Date: Mar. 20, 2024

(30) Foreign Application Priority Data
Sep. 27, 2021 (KR) 10-2021-0127170

(51) Int. Cl. A61B 5/00; A61B 5/256; A61B 5/291; A61B 5/374

(52) U.S. Cl. CPC A61B 5/7267; A61B 5/256; A61B 5/291; A61B 5/374; A61B 5/7225; A61B 5/7257; A61B 5/742; A61B 2560/045; A61B 2560/0468

(57) ABSTRACT

The present invention provides a method for providing information on a major depressive disorder implemented by a processor. Provided are a method for providing information on a major depressive disorder and a device using the same, the method comprising the steps of: receiving brain wave data of a subject; extracting feature data of at least one of power spectrum densities (PSDs), a functional connectivity, and a network index with respect to the brain wave data; and determining whether the subject has a major depressive disorder on the basis of at least one feature data, by using a classification model trained to output whether a subject has a major depressive disorder on the basis of the at least one feature data as an input, wherein the subject is a subject suspected of suffering from a major depressive disorder without having a history of drug use.

### RELATED APPLICATIONS, INCORPORATIONS BY REFERENCE, AND CLAIMS OF PRIORITY

This application claims the benefit of priority to PCT Application No. PCT/KR2022/014471 entitled “METHOD FOR PROVIDING INFORMATION OF MAJOR DEPRESSIVE DISORDERS AND DEVICE FOR PROVIDING INFORMATION OF MAJOR DEPRESSIVE DISORDERS USING THE SAME,” filed on Sep. 27, 2022, which claims priority to Korean National Application No. 10-2021-0127170 entitled “METHOD FOR PROVIDING INFORMATION OF MAJOR DEPRESSIVE DISORDERS AND DEVICE FOR PROVIDING INFORMATION OF MAJOR DEPRESSIVE DISORDERS USING THE SAME,” filed on Sep. 27, 2021. All the aforementioned applications and patents are hereby incorporated by reference in their entirety.

### TECHNICAL FIELD

The present invention relates to a method for providing information on a major depressive disorder and a device for providing information on a major depressive disorder using the same, and more particularly, to a method for providing information on a major depressive disorder which provides information on whether a major depressive disorder occurs on the basis of brain wave data and a device for providing information on a major depressive disorder using the same.

### BACKGROUND ART

A mental disorder may refer to a dysfunction in psychology or behaviors. At this time, the major depressive disorder may be caused by genetic causes, physical and organic causes, and mental and psychological causes such as a stress.

In particular, the major depressive disorder, which is one of the mental disorders, usually develops slowly and may cause symptoms such as insomnia, sad feelings, obsession with the past, distraction, hopelessness, fatigue, and loss of appetite due to hyperactivity, separation anxiety disorder, or intermittent depressive symptoms over several years.

The prevalence of major depressive disorder is increasing in the modern society as the frequency of exposure to mental stress increases. However, the major depressive disorder has many similar symptoms which are shared with other major depressive orders and a degree thereof varies from person to person, which makes it difficult to distinguish accurately.

As described above, the development of optimal classification criteria for major depressive disorder may be important for accurate diagnosis of the major depressive disorder.

Accordingly, development for a new diagnosis criterion and system of major depressive disorders which may improve the accuracy of the diagnosis is consistently demanded.

### DETAILED DESCRIPTION OF THE INVENTION

### Technical Problems

In the meantime, in order to clearly diagnose the major depressive disorder, a functional magnetic resonance imaging (fMRI) on the basis of a dynamic neural activity which represents a unique characteristic of each disorder has emerged.

To be more specific, according to fMRI analysis, a patient with a major depressive disorder may show different neural responses from normal subjects when reading emotional texts. Therefore, the fMRI analysis result may be provided as information for accurate diagnosis of a major depressive disorder.

In the meantime, in the case of the fMRI, the patients may complain of anxiety or fear during the diagnosis process. Moreover, when the fMRI is applied to the diagnosis of major depressive disorders, the fMRI still has many limitations such as expensive analysis costs, spatial and temporal restrictions.

Specifically, fMRI focuses only on the neural activity while processing the emotional information but does not consider important pathologies such as an altered cognitive process so that there may be limitations in providing reliable information for accurate diagnosis of the major depressive disorder.

In the meantime, the inventors of the present invention have paid attention that with regard to the major depressive disorder, changes in biosignals will precede as a part of the human body's response.

Specifically, the inventors of the present invention have paid attention to the change in brain wave data with regard to the onset of the major depressive disorder and have recognized that the above-described problem of the fMRI analysis may be overcome using the brain wave data.

To be more specific, the inventors of the present invention may recognize that it is possible to extract a feature associated with the major depressive disorder from a brain wave signal and classify a major depressive disorder, specifically, whether a subject suspected of suffering from a major depressive disorder without having a history of drug use has a major depressive disorder with a high reliability.

As a result, the inventors of the present invention could develop a system for providing information on a major depressive disorder on the basis of a brain wave signal.

Further, the inventors of the present invention could recognize that the application of brain wave data which may be acquired from a sensor of the brain wave signal and/or brain activity data of a source activity which is activated can contribute to the accurate diagnosis of the major depressive disorder.

Moreover, the inventors of the present invention may apply a classification model which is trained by the brain activity data to predict the major depressive disorder to the information providing system to provide information with a high reliability.

Accordingly, the inventors of the present invention tried to reduce the number of brain wave channels which are used as learning data while maintaining a classification performance of the classification model on whether a subject has a major depressive disorder.

As a result, a classification model configured to classify whether a subject has a major depressive disorder with a high accuracy even with a reduced number of channels was constructed and the inventors of the present invention tried to apply the classification model to the information providing system.

Therefore, the inventors of the present invention recognized that brain wave data (or feature data) acquired from the reduced number of channels is used to solve the problem of overfitting which is generated in a model which uses all brain wave data as a feature parameter.

Accordingly, an object to be achieved by the present invention is to provide a method for providing information on a major depressive disorder and a device thereof configured to determine a feature from brain wave data and/or brain activity data acquired from the subject and determine whether a major depressive disorder occurs in the subject using a classification model.

Objects of the present invention are not limited to the above-mentioned objects, and other objects, which are not mentioned above, can be clearly understood by those skilled in the art from the following descriptions.

### Technical Solution

In order to achieve the above-described objects, a method for providing information on a major depressive disorder according to an exemplary embodiment of the present invention is provided. The method for providing information according to the exemplary embodiment of the present invention is a method for providing information on a major depressive disorder implemented by a processor including receiving brain wave data of a subject; extracting at least one feature data of power spectrum densities (PSDs), a functional connectivity, and a network index with respect to the brain wave data; and determining whether the subject has a major depressive disorder on the basis of at least one feature, by using a classification model trained to output whether a subject has a major depressive disorder on the basis of the at least one feature data as an input. At this time, the subject is a subject suspected of suffering from a major depressive disorder without having a history of drug use.

According to a feature of the present invention, receiving brain wave data may include receiving brain wave data of a subject measured from a plurality of electrode channels selected from FP1, FPZ, FP2, AF3, AF4, F7, F5, F3, F1, FZ, F2, F4, F6, F8, FT7, FC5, FC3, FC1, FCZ, FC2, FC4, FC6, FT8, T7, C5, C3, C1, CZ, C2, C4, C6, T8, TP7, CP5, CP3, CP1, CPZ, CP2, CP4, CP6, TP8, P7, P5, P3, P1, PZ, P2, P4, P6, P8, PO7, PO5, PO3, POZ, PO4, PO6, PO8, CB1, O1, OZ, O2, and CB2. Further, extracting at least one feature data may include: extracting at least one feature data from a plurality of frequency bands selected from low-alpha, high-alpha, low-beta, high-beta, gamma, delta, and theta of brain wave data measured from the plurality of electrode channels.

According to another feature of the present invention, the plurality of electrode channels may be a first electrode channel set of FP1, FPZ, FP2, AF3, AF4, F7, F5, F3, F1, FZ, F2, F4, F6, F8, FT7, FC5, FC3, FC1, FCZ, FC2, FC4, FC6, FT8, T7, C5, C3, C1, CZ, C2, C4, C6, T8, TP7, CP5, CP3, CP1, CPZ, CP2, CP4, CP6, TP8, P7, P5, P3, P1, PZ, P2, P4, P6, P8, PO7, PO5, PO3, POZ, PO4, PO6, PO8, CB1, O1, OZ, O2, and CB2, a second electrode channel set of FP1, FP2, F7, F3, FZ, F4, F8, FT7, FC3, FCZ, FC4, FT8, T7, C3, CZ, C4, T8, TP7, CP3, CPZ, CP4, TP8, P7, P3, PZ, P4, P8, O1, OZ, and O2, a third electrode channel set of FP1, FP2, F7, F3, FZ, F4, F8, T7, C3, CZ, C4, T8, P7, P3, PZ, P4, P8, O1, and O2, or a fourth electrode channel set of F3, F4, T7, C3, C4, T8, P3, P4, O1, and O2. Further, the plurality of frequency bands may be a first frequency band set of theta, low-alpha, and high-alpha, a second frequency band set of delta and theta, a third frequency band set of delta, theta, low-alpha, high-alpha, and low-beta, or a fourth frequency band set of theta and low-beta.

According to still another feature of the present invention, the plurality of electrode channels may be the first electrode channel set and the plurality of frequency bands may be the first frequency band set.

According to still another feature of the present invention, the plurality of electrode channels may be the second electrode channel set and the plurality of frequency bands may be the second frequency band set.

According to still another feature of the present invention, the plurality of electrode channels may be the third electrode channel set and the plurality of frequency bands may be the third frequency band set.

According to still another feature of the present invention, the plurality of electrode channels may be the fourth electrode channel set and the plurality of frequency bands may be the fourth frequency band set.

According to still another feature of the present invention, at least one feature data may be a functional connectivity. At this time, extracting at least one feature data may include: determining a connectivity of a phase locking value (PLV) for the brain wave data.

According to still another feature of the present invention, at least one feature data may be a functional connectivity and a network index. Further, extracting at least one feature data may include: extracting the functional connectivity from the brain wave data; and determining the network index based on network structural feature data of the functional connectivity.

According to still another feature of the present invention, determining a network index may include determining at least one index of a strength for the functional connectivity, a clustering coefficient, and a path length.

According to still another feature of the present invention, the information providing method may further include: generating brain activity data based on the brain wave data after receiving brain wave data. At this time, extracting at least one feature data may further include: extracting at least one feature data for each of brain wave data and brain activity data. Further, the determining of whether the subject has a major depressive disorder may further include determining whether the subject has a major depressive disorder using a classification model based on feature data for each of brain wave data and brain activity data.

According to still another feature of the present invention, the information providing method may further include: filtering the brain activity data based on a band pass filter, which is performed after generating the brain activity data.

According to still another feature of the present invention, the brain wave data may be defined as brain wave data acquired in a resting state.

In order to achieve the above-described objects, a device for providing information on a major depressive disorder according to an exemplary embodiment of the present invention is provided.

At this time, the information providing device includes a communication unit configured to receive brain wave data of a subject; and a processor connected to communicate with the communication unit. The processor is configured to extract at least one feature of power spectrum densities (PSDs), a functional connectivity, and a network index with respect to the brain wave data, and determine whether the subject has a major depressive disorder on the basis of at least one feature, by using a classification model trained to output whether a subject has a major depressive disorder on the basis of the at least one feature data as an input. Furthermore, the subject is a subject suspected of suffering from a major depressive disorder without having a history of drug use.

According to a feature of the present invention, the communication unit may be configured to receive brain wave data of a subject measured from a plurality of electrode channels selected from FP1, FPZ, FP2, AF3, AF4, F7, F5, F3, F1, FZ, F2, F4, F6, F8, FT7, FC5, FC3, FC1, FCZ, FC2, FC4, FC6, FT8, T7, C5, C3, C1, CZ, C2, C4, C6, T8, TP7, CP5, CP3, CP1, CPZ, CP2, CP4, CP6, TP8, P7, P5, P3, P1, PZ, P2, P4, P6, P8, PO7, PO5, PO3, POZ, PO4, PO6, PO8, CB1, O1, OZ, O2, and CB2. At this time, the processor may be configured to extract at least one feature data from a plurality of frequency bands selected from low-alpha, high-alpha, low-beta, high-beta, gamma, delta, and theta of brain wave data measured from the plurality of electrode channels.

According to another feature of the present invention, the plurality of electrode channels may be a first electrode channel set of FP1, FPZ, FP2, AF3, AF4, F7, F5, F3, F1, FZ, F2, F4, F6, F8, FT7, FC5, FC3, FC1, FCZ, FC2, FC4, FC6, FT8, T7, C5, C3, C1, CZ, C2, C4, C6, T8, TP7, CP5, CP3, CP1, CPZ, CP2, CP4, CP6, TP8, P7, P5, P3, P1, PZ, P2, P4, P6, P8, PO7, PO5, PO3, POZ, PO4, PO6, PO8, CB1, O1, OZ, O2, and CB2, a second electrode channel set of FP1, FP2, F7, F3, FZ, F4, F8, FT7, FC3, FCZ, FC4, FT8, T7, C3, CZ, C4, T8, TP7, CP3, CPZ, CP4, TP8, P7, P3, PZ, P4, P8, O1, OZ, and O2, a third electrode channel set of FP1, FP2, F7, F3, FZ, F4, F8, T7, C3, CZ, C4, T8, P7, P3, PZ, P4, P8, O1, and O2, or a fourth electrode channel set of F3, F4, T7, C3, C4, T8, P3, P4, O1, and O2. Further, the plurality of frequency bands may be a first frequency band set of theta, low-alpha, and high-alpha, a second frequency band set of delta and theta, a third frequency band set of delta, theta, low-alpha, high-alpha, and low-beta, or a fourth frequency band set of theta and low-beta.

According to another feature of the present invention, the plurality of electrode channels may be the first electrode channel set and the plurality of frequency bands may be the first frequency band set.

According to still another feature of the present invention, the plurality of electrode channels may be the second electrode channel set and the plurality of frequency bands may be the second frequency band set.

According to still another feature of the present invention, the plurality of electrode channels may be the third electrode channel set and the plurality of frequency bands may be the third frequency band set.

According to still another feature of the present invention, the plurality of electrode channels may be the fourth electrode channel set and the plurality of frequency bands may be the fourth frequency band set.

According to still another feature of the present invention, the at least one feature data is a functional connectivity, and the processor may be further configured to determine a connectivity of a phase locking value (PLV) for the brain wave data.

According to still another feature of the present invention, the at least one feature data is a functional connectivity and a network index, and the processor may be further configured to extract a functional connectivity from the brain wave data and determine a network index based on network structural feature data of the functional connectivity.

According to still another feature of the present invention, the processor may be further configured to determine at least one index of a strength for the functional connectivity, a clustering coefficient, and a path length.

According to still another feature of the present invention, the processor may be further configured to filter the brain wave data based on a band pass filter.

Other detailed matters of the exemplary embodiments are included in the detailed description and the drawings.

### Effects of the Invention

According to the present invention, a providing system configured to extract features from brain wave data and/or brain activity data of source activity, acquired from a sensor of a brain wave signal, and classify whether a major depressive disorder has occurred based on the feature is provided to contribute to a reliable diagnosis of a major depressive disorder.

Therefore, according to the present invention, it is possible to overcome the limitation of the analysis method, such as fMRI, having still a lot of limitations, such as high analysis costs, spatial and temporal restrictions while providing less reliable information.

Moreover, the present invention may provide an information providing system which applies a classification model trained by feature data extracted from the brain wave data and/or brain activity data to predict whether a subject has a major depressive disorder to provide information with a high reliability about the onset of the major depressive disorder.

Specifically, the present invention uses a classification model configured to classify whether the subject has a major depressive disorder with a high accuracy even with a reduced number of channels to solve the problem of overfitting which is caused in a model which uses all brain wave data as a feature parameter.

Specifically, according to the present invention, the computational efficiency may be improved by reducing computational cost and time and reducing an overfitting possibility through a channel reduction strategy.

Further, during the process of acquiring and measuring brain wave data, the number of brain wave channels is minimized to reduce a load of a user.

That is, the users may easily acquire information on their own mental health without temporal and spatial restrictions. Further, medical staff may acquire information on suspected subjects to consistently monitor the subjects suspected of suffering from a major depressive disorder.

Therefore, the present invention may contribute to early diagnosis and good treatment prognosis of the major depressive disorder by providing information on whether major depressive disorder has occurred.

The effects according to the present invention are not limited to the contents exemplified above, and more various effects are included in the present invention.

### BRIEF DESCRIPTION OF DRAWINGS

FIG. 1A is a schematic diagram for explaining a system for providing information on a major depressive disorder using biosignal data according to an exemplary embodiment of the present invention.

FIG. 1B is a schematic diagram for explaining a device for providing information on a major depressive disorder according to an exemplary embodiment of the present invention.

FIG. 1C is a schematic diagram for explaining a user mobile device which is provided with information from a device for providing information on a major depressive disorder according to an exemplary embodiment of the present invention.

FIG. 2 is a schematic flowchart for explaining a method for determining whether a major depressive disorder has occurred, on the basis of brain activity data of a subject in a device for providing information on a major depressive disorder according to an exemplary embodiment of the present invention.

FIG. 3A illustrates an electrode channel set for receiving brain wave data in a method for providing information on a major depressive disorder according to an exemplary embodiment of the present invention.

FIG. 3B illustrates a procedure of a method for providing information on a major depressive disorder according to an exemplary embodiment of the present invention.

FIG. 3C illustrates a step of extracting features from brain wave data and/or brain activity data in a method for providing information on a major depressive disorder according to an exemplary embodiment of the present invention.

FIGS. 4A to 4C illustrate an evaluation result according to a feature data type of a classification model which is applied to a device for providing information on a major depressive disorder, according to an exemplary embodiment of the present invention.

FIGS. 5A to 5D illustrate an evaluation result according to a brain wave data type of a classification model which is applied to a device for providing information on a major depressive disorder, according to an exemplary embodiment of the present invention.

### BEST MODES FOR CARRYING OUT THE INVENTION

The present invention will become clear by referring to the exemplary embodiments described in detail below together with the drawings. However, the present invention is not limited to the following exemplary embodiments but may be implemented in various different forms. The exemplary embodiments are provided only to complete the disclosure of the present invention and to fully provide a person having ordinary skill in the art to which the present invention pertains with the category of the invention. In the description of drawings, like reference numerals denote like components.

In this specification, the expression “have”, “may have”, “include”, or “may include” represent the presence of the characteristic (for example, a numerical value, a function, an operation, or a component such as a part”), but do not exclude the presence of additional characteristic.

In the specification, the expression “A or B”, “at least one of A or/and B”, or “at least one or more of A or/and B” may include all possible combinations of enumerated items. For example, the terms “A or B”, “at least one of A and B”, or “at least one of A or B” may refer to an example which includes (1) at least one A, (2) at least one B, or (3) all at least one A and at least one B.

Although the expressions used in the present specification “first”, “second”, and the like, may be used herein to describe various components regardless of an order and/or importance, these terms are only used to distinguish one component from another, but the components are not limited by these terms. For example, a first user device and a second user device may refer to different user devices regardless of the order or the importance. For example, without departing from the scope of the present specification, a first component may be referred to as a second component, and similarly, a second component may be referred to as a first component.

When a component (for example, a first component) is referred to as being “(operatively or communicatively) coupled with/to” or “connected to” another component (for example, a second component), it can be understood that the component is directly connected to the other component or connected to the other component via another component (for example, a third component). In contrast, when a component (for example, a first component) is referred to as being “directly coupled with/to” or “directly connected to” another component (for example, a second component), it can be understood that there is not another component (for example, a third component) between the components.

The expression used in the present specification “configured to (or set to)” may be interchangeably used with, for example, “suitable for”, “having the capacity to”, “designed to”, “adapted to”, “made to”, or “capable of” depending on the situation. The terms “configured to (or set)” may not necessarily mean only “specifically designed to” in a hardware manner. Instead, in some situations, the term “a device configured to” may mean that the device is “capable of” something together with another device or components. For example, the terms “a processor configured (or set) to perform A, B, and C” may refer to a dedicated processor (for example, an embedded processor) configured to perform the corresponding operation or a generic-purpose processor (for example, a CPU or an application processor) which is capable of performing the operations by executing one or more software programs stored in a memory device.

The terms used in this specification are merely used to describe a specific exemplary embodiment, but do not intend to limit the scope of another exemplary embodiment. A singular form may include a plural form if there is no clearly opposite meaning in the context. Terms used herein including technical or scientific terms may have the same meaning as commonly understood by those skilled in the art described in this specification. Among the terms used in this specification, terms defined in the general dictionary may be interpreted as having the same or similar meaning as the meaning in the context of the related art but are not ideally or excessively interpreted to have formal meanings unless clearly defined in this specification. In some cases, even though the terms are defined in this specification, the terms are not interpreted to exclude the exemplary embodiments of the present specification.

The features of various exemplary embodiments of the present invention can be partially or entirely bonded to or combined with each other and can be interlocked and operated in technically various ways fully understood by those skilled in the art, and the exemplary embodiments can be carried out independently of or in association with each other.

For clarity of interpretation of the present specification, terms used in the present specification will be defined below.

The term used in the present specification, “major depressive disorder” is a mood disorder which refers to a mental disorder which experiences one or more episodes of major depressive disorder without a manic or hypomanic episode.

In the meantime, in the specification of the present invention, the major depressive disorder may include “major depressive disorder” and even “depression”.

The term used in the present specification “subject” may be a subject suspected of suffering from a major depressive disorder. Desirably, the subject in the present specification may refer to a subject without having a history of drug use. More desirably, the subject in the present specification may refer to a woman without having a history of drug use. However, it is not limited thereto.

The term used in the present specification “brain wave data” may refer to an electroencephalogram (EEG) signal value recorded in a sensor which senses a brain wave. To be more specific, the brain wave data may be acquired by measuring an electrical signal generated from the brain, from the two or more electrode channels.

In the meantime, the brain wave data may be a signal, or a signal value acquired from a sensor so that in the present specification, the brain wave data may be interpreted as having the same meaning as sensor data.

According to the feature of the present invention, the brain wave data may include brain wave data measured from a plurality of electrode channels selected from FP1, FPZ, FP2, AF3, AF4, F7, F5, F3, F1, FZ, F2, F4, F6, F8, FT7, FC5, FC3, FC1, FCZ, FC2, FC4, FC6, FT8, T7, C5, C3, C1, CZ, C2, C4, C6, T8, TP7, CP5, CP3, CP1, CPZ, CP2, CP4, CP6, TP8, P7, P5, P3, P1, PZ, P2, P4, P6, P8, PO7, PO5, PO3, POZ, PO4, PO6, PO8, CB1, O1, OZ, O2, and CB2.

According to the feature of the present invention, the brain wave data may be brain wave data acquired from a first electrode channel set of FP1, FPZ, FP2, AF3, AF4, F7, F5, F3, F1, FZ, F2, F4, F6, F8, FT7, FC5, FC3, FC1, FCZ, FC2, FC4, FC6, FT8, T7, C5, C3, C1, CZ, C2, C4, C6, T8, TP7, CP5, CP3, CP1, CPZ, CP2, CP4, CP6, TP8, P7, P5, P3, P1, PZ, P2, P4, P6, P8, PO7, PO5, PO3, POZ, PO4, PO6, PO8, CB1, O1, OZ, O2, and CB2, a second electrode channel set of FP1, FP2, F7, F3, FZ, F4, F8, FT7, FC3, FCZ, FC4, FT8, T7, C3, CZ, C4, T8, TP7, CP3, CPZ, CP4, TP8, P7, P3, PZ, P4, P8, O1, OZ, and O2, a third electrode channel set of FP1, FP2, F7, F3, FZ, F4, F8, T7, C3, CZ, C4, T8, P7, P3, PZ, P4, P8, O1, and O2, or a fourth electrode channel set of F3, F4, T7, C3, C4, T8, P3, P4, O1, and O2.

According to the feature of the present invention, the brain wave data may be time-series brain wave data acquired in a resting state in which stimulation is not applied to the subject.

The term used in the present specification “brain activity data” may refer to data of source activity which is activated while the stimulus is output. At this time, the source activity may correspond to a current source density (CSD) for the brain active area.

For example, the brain activity data may include a current source density (CSD) or a source activity in at least one brain region of banks of the superior temporal sulcus, caudal anterior cingulate, caudal middle frontal, cuneus, entorhinal, frontal pole, fusiform, inferior parietal, inferior temporal, insula, isthmus cingulate, lateral occipital, lateral orbitofrontal, lingual, medial orbitofrontal, middle temporal, para central, para hippocampal, pars opercularis, pars orbitalis, pars triangularis, pericalcarine, post central, posterior cingulate, precentral, precuneus, rostral anterior cingulate, rostral middle frontal, superior frontal, superior parietal, superior temporal, supramarginal, temporal pole, and transverse temporal. However, it is not limited thereto.

As the brain activity data is defined as a source activity, the brain activity data may be interpreted as the same meaning as source data in the present specification.

In the meantime, the brain activity data may be generated on the basis of the above-described brain wave data.

For example, the brain activity data may be acquired by estimating a source activity for a voxel corresponding to a source space of the brain wave data using at least one of low-resolution brain electromagnetic tomography (LORETA), standardized low-resolution brain electromagnetic tomography (sLORETA), exact resolution brain electromagnetic tomography (eLORETA), minimum-norm estimate (MNE), weighted MNE (wMNE), dynamic statistical parametric mapping (dSPM), linearly constrained minimum variance (LCMV) beamformers Programs-LORETA/sLORETA toolbox, brainstrom, eConnectome, fieldtrip, and EEGlab.

The term used in the present specification “frequency band” refers to a frequency region of a brain wave, and more specifically, the frequency band of the brain wave data may be a delta wave δ of 1 to 4 Hz, a theta wave θ of 4 to 8 Hz, an alpha wave α of 8 to 12 Hz, a beta wave β of 12 to 30 Hz, or a gamma wave γ of 30 to 35 Hz.

However, it is not limited thereto, and the brain wave may be present in the frequency region of low-alpha of 8 to 10 Hz, high-alpha of 10 to 12 Hz, low-beta of 12 to 22 Hz, or high-beta of 22 to 30 Hz.

In the meantime, in the specification of the present invention, the frequency band may refer to a plurality of frequency bands.

To be more specific, the plurality of frequency bands may include a first frequency band set of theta, low-alpha, and high-alpha, a second frequency band set of delta and theta, a third frequency band set of delta, theta, low-alpha, high-alpha, and low-beta, or a fourth frequency band set of theta and low-beta. However, it is not limited thereto.

The term used in the present specification “feature data” may refer to data extracted based on a functional connectivity and a network system of brain wave data (furthermore, brain activity data). At this time, the feature data in the present specification may be interchangeably used with a feature.

In the meantime, the feature data may be at least one of power spectrum densities (PSDs), a functional connectivity, and a network index.

Desirably, the feature data may be a functional connectivity, and specifically, a functional connectivity of brain wave data. More desirably, the feature data may be a phase locking value (PLV) of the brain wave data. To be more specific, the feature data may be PLV of the frequency region corresponding to a first frequency band set of theta, low-alpha, and high-alpha, a second frequency band set of delta and theta, a third frequency band set of delta, theta, low-alpha, high-alpha, and low-beta, or a fourth frequency band set of theta and low-beta. However, it is not limited thereto.

For example, the feature data of the PLV which is a statistical value for investigating task-induced changes in long-distance synchronization of the neural activity may be determined from the brain wave data and the brain activity data.

The term used in the present specification “classification model” may refer to a model trained to classify the major depressive disorder, based on feature data extracted from brain wave data and/or brain activity data of a subject.

According to the feature of the present invention, the classification model may be a model configured to output the major depressive disorder or normal based on the feature data as an input.

For example, the classification model may be further configured to output 0 or 1 depending on whether a subject has a major depressive disorder.

In the meantime, the classification model is not limited thereto, but may be configured to output more various classes depending on the severity of the major depressive disorder.

The classification model may be a model on the basis of at least one algorithm of a support vector machine (SVM), a decision tree, a random forest, adaptive boosting (AdaBoost), and penalized logistic regression (PLR). However, the classification model of the present invention is not limited thereto and may be provided on the basis of various learning algorithms.

For example, the classification model may be a model which is trained to classify normal or major depressive disorder, based on feature data having high contribution to classification of normal and major depressive disorder, selected based on a Fisher score for a plurality of feature data.

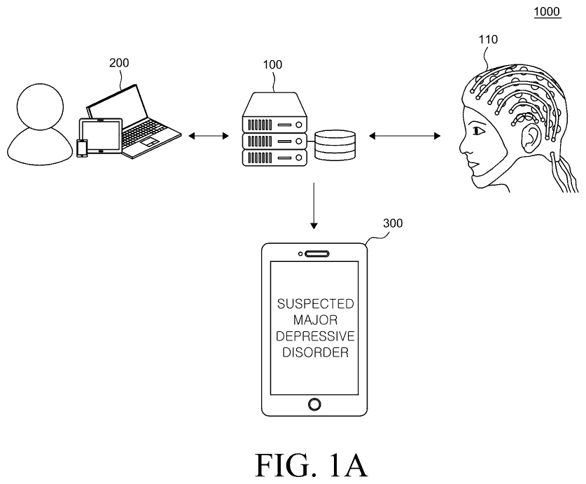

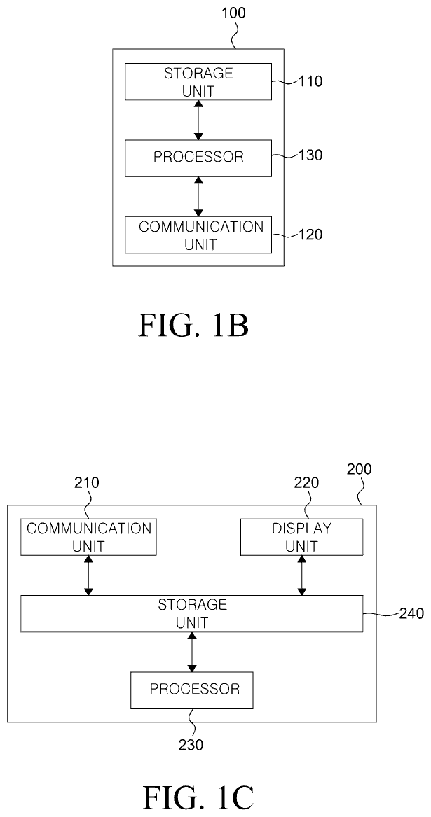

Hereinafter, the device for providing information on a major depressive disorder according to various exemplary embodiments of the present invention will be described in detail with reference to FIGS. 1A to 1C.

FIG. 1A is a schematic diagram for explaining a system for providing information on a major depressive disorder using biosignal data according to an exemplary embodiment of the present invention.

First, referring to FIG. 1A, an information providing system 1000 may be a system configured to provide information related to a major depressive disorder based on a brain wave of a user. At this time, the system 1000 for providing information on a major depressive disorder may include a device 100 for providing information on a major depressive disorder configured to determine whether a major depressive disorder occurs in a subject, on the basis of brain wave data and/or brain activity data, a user mobile device 200, a medical staff device 300, and a brain wave measuring device 400 configured to be in close contact with a user's scalp to measure a brain wave.

First, the device 100 for providing information on a major depressive disorder may include a general-purpose computer, a laptop, and/or a data server, etc. which performs various computations to evaluate whether a major depressive disorder occurs, based on a brain wave of a user which is provided from the brain wave measuring device 400. At this time, the user mobile device 200 may be a device which accesses a web server which provides a web page for a major depressive disorder or a mobile web server which provides a mobile web site but is not limited thereto. Further, the brain wave measuring device 400 may be configured by a plurality of electrode channels configured to enclose a head of the user from the outside. The plurality of electrode channels may be a first electrode channel set of FP1, FPZ, FP2, AF3, AF4, F7, F5, F3, F1, FZ, F2, F4, F6, F8, FT7, FC5, FC3, FC1, FCZ, FC2, FC4, FC6, FT8, T7, C5, C3, C1, CZ, C2, C4, C6, T8, TP7, CP5, CP3, CP1, CPZ, CP2, CP4, CP6, TP8, P7, P5, P3, P1, PZ, P2, P4, P6, P8, PO7, PO5, PO3, POZ, PO4, PO6, PO8, CB1, O1, OZ, O2, and CB2, a second electrode channel set of FP1, FP2, F7, F3, FZ, F4, F8, FT7, FC3, FCZ, FC4, FT8, T7, C3, CZ, C4, T8, TP7, CP3, CPZ, CP4, TP8, P7, P3, PZ, P4, P8, O1, OZ, and O2, a third electrode channel set of FP1, FP2, F7, F3, FZ, F4, F8, T7, C3, CZ, C4, T8, P7, P3, PZ, P4, P8, O1, and O2, or a fourth electrode channel set of F3, F4, T7, C3, C4, T8, P3, P4, O1, and O2.

Specifically, the device 100 for providing information on a major depressive disorder receives brain wave data from the brain wave measuring device 400 and extracts a feature from the received brain wave data to be configured to classify as a major depressive disorder or normal.

The device 100 for providing information on a major depressive disorder may supply data analyzing whether a major depressive disorder occurs in a subject to the user mobile device 200 and the medical staff device 300.

Data provided from the device 100 for providing information on a major depressive disorder may be provided to a web page through a web browser installed in the user mobile device 200 and/or the medical staff device 300 or provided as an application or a program. In various exemplary embodiments, the data may be provided to be included in a platform in a client-server environment.

Next, the user mobile device 200 is an electronic device which provides a user interface to request information about whether a major depressive disorder occurs in a subject and displays analysis result data and may include at least one of a smart phone, a tablet personal computer (PC), a notebook and/or a PC, etc.

The user mobile device 200 may receive an analysis result of onset of a major depressive disorder of a subject from the device 100 for providing information on a major depressive disorder and display the received result on a display unit of the user mobile device 200. Here, the analysis result may include whether a major depressive disorder has occurred, a high, moderate, or low risk level of onset of a major depressive disorder, an onset probability, and the like.

Next, referring to FIG. 1B, a component of the device 100 for providing information on a major depressive disorder of the present invention will be described in detail.

FIG. 1B is a schematic diagram for explaining a device for providing information on a major depressive disorder according to an exemplary embodiment of the present invention.

Referring to FIG. 1B, the device 100 for providing information on a major depressive disorder includes a storage unit 110, a communication unit 120, and a processor 130.

First, the storage unit 110 may store various data for evaluating whether a major depressive disorder has occurred in a subject. According to various exemplary embodiments, the storage unit 110 may include at least one type of storage medium of a flash memory type, a hard disk type, a multimedia card micro type, a card type memory (for example, an SD or XD memory, and the like), a RAM, an SRAM, a ROM, an EEPROM, a PROM, a magnetic memory, a magnetic disk, and an optical disk.

The communication unit 120 communicatively connects the device 100 for providing information on a major depressive disorder with the external device. The communication unit 120 is connected to the user mobile device 200, the medical staff device 300, and the brain wave measuring device 400 using wired/wireless communication to transmit and receive various data. Specifically, the communication unit 120 may receive brain wave data of a subject from the brain wave measuring device 400 and receive brain activity data from a brain electromagnetic tomography (not illustrated). Further, the communication unit 120 may transmit an analysis result to the user mobile device 200 and/or the medical staff device 300.

The processor 130 is operatively connected to the storage unit 110 and the communication unit 120 and may perform various instructions to analyze the brain wave data and/or the brain activity data for the subject.

Specifically, the processor 130 receives brain wave data of a subject from the brain wave measuring device 400 through the communication unit 120, generates brain activity data based on the received brain wave data, and extracts a feature to evaluate an onset risk level of a major depressive disorder of a subject.

In the meantime, the processor 130 may be configured to convert the brain wave data into the brain activity data using at least one of low-resolution brain electromagnetic tomography (LORETA), standardized low-resolution brain electromagnetic tomography (sLORETA), exact resolution brain electromagnetic tomography (eLORETA), minimum-norm estimate (MNE), weighted MNE (wMNE), dynamic statistical parametric mapping (dSPM), linearly constrained minimum variance (LCMV) beamformers Programs-LORETA/sLORETA toolbox, brainstrom, eConnectome, fieldtrip, and EEGlab.

Moreover, the processor 130 may be based on a classification model configured to classify whether a major depressive disorder has occurred based on feature data extracted from the brain wave data and/or the brain activity data.

For example, the processor 130 may be based on a classification model configured to classify whether a major depressive disorder has occurred based on at least one feature data of power spectrum densities (PSDs), a functional connectivity, and a network index extracted from the brain wave data and/or the brain activity data.

Therefore, the processor 130 of the classification model may provide a highly reliable analysis result about whether a major depressive disorder has occurred.

The user may easily acquire information on his/her own mental health without having temporal and spatial restrictions using the user mobile device 200. Further, medical staff may acquire information about subjects from the medical staff device 300 to consistently monitor the subjects suspected of suffering from a major depressive disorder.

As described above, the present invention may contribute to early diagnosis and good treatment prognosis of major depressive disorder by classifying whether a major depressive disorder has occurred with a high accuracy and providing information thereof.

In the meantime, referring to FIG. 1C, the user mobile device 200 includes a communication unit 210, a display unit 220, a storage unit 230, and a processor 240.

The communication unit 210 connects the user mobile device 200 to communicate with the external device. The communication unit 210 is connected to the device 100 for providing information on a major depressive disorder using wired/wireless communication to transmit and receive various data. Specifically, the communication unit 210 may receive an analysis result associated with diagnosis of the major depressive disorder of the subject from the device 100 for providing information on a major depressive disorder.

The display unit 220 may display various interface screens to represent an analysis result associated with the diagnosis of the major depressive disorder of the subject.

According to various exemplary embodiments, the display unit 220 may include a touch screen and for example, may receive touch, gesture, proximity, drag, swipe, or hovering input which uses an electronic pen or a part of a body of the user.

The storage unit 230 may store various data used to provide a user interface to represent result data. According to various exemplary embodiments, the storage unit 230 may include at least one type of storage medium of flash memory type, hard disk type, multimedia card micro type, card type memories (for example, ab SD or XD memory and the like), a random access memory (RAM), a static random access memory (SRAM), a read only memory (ROM), an electrically erasable programmable read-only memory (EEPROM), a programmable read-only memory (PROM), a magnetic memory, a magnetic disk, and an optical disk.

The processor 240 is operatively connected to the communication unit 210, the display unit 220, and the storage unit 230 and may perform various instructions to provide a user interface to represent result data.

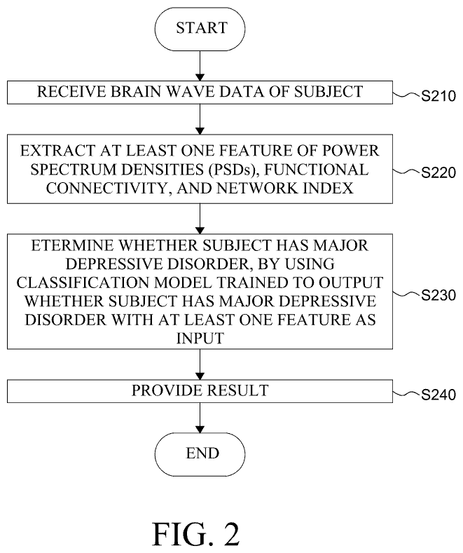

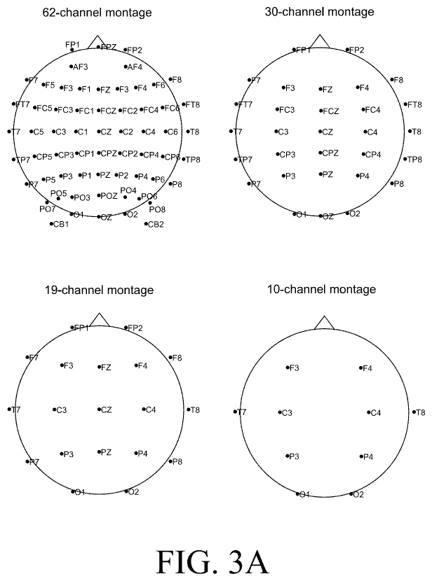

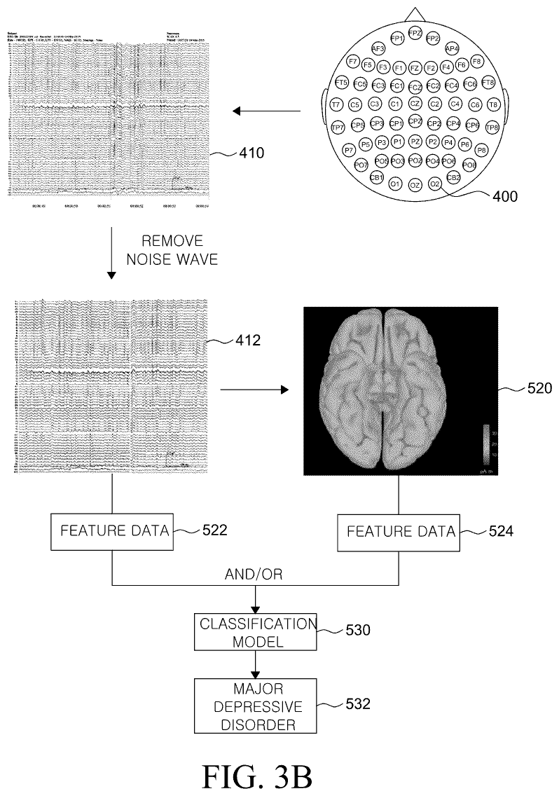

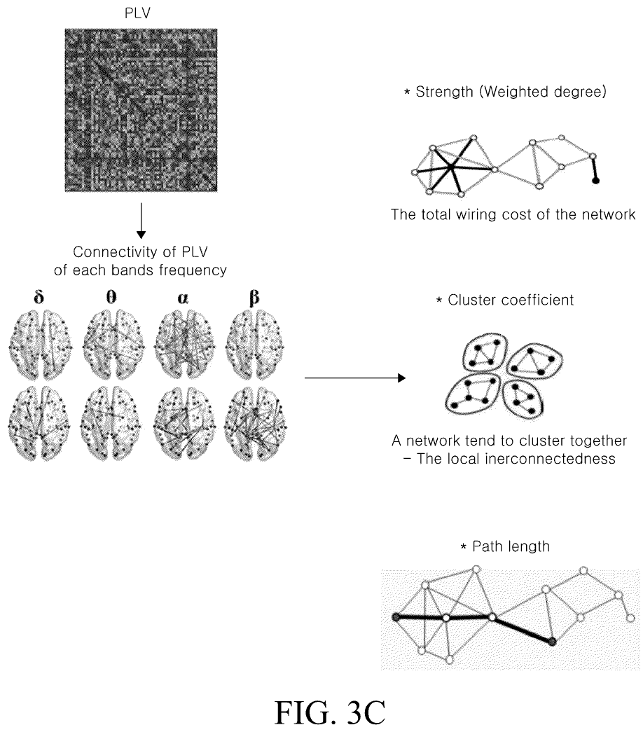

Hereinafter, an information providing method according to various exemplary embodiments of the present invention will be described with reference to FIGS. 2, 3A to 3C.

FIG. 2 is a schematic flowchart for explaining a method for determining whether a major depressive disorder has occurred, on the basis of brain activity data of a subject in a device for providing information on a major depressive disorder according to an exemplary embodiment of the present invention. FIG. 3A illustrates an electrode channel set for receiving brain wave data in a method for providing information on a major depressive disorder according to an exemplary embodiment of the present invention. FIG. 3B illustrates a procedure of a method for providing information on a major depressive disorder according to an exemplary embodiment of the present invention. FIG. 3C illustrates a step of extracting features from brain wave data and/or brain activity data in a method for providing information on a major depressive disorder according to an exemplary embodiment of the present invention.

First, referring to FIG. 2, brain wave data of a subject is received according to a method for providing information on a major depressive disorder according to an exemplary embodiment of the present invention (S210). Next, at least one feature data of power spectrum densities (PSDs), a functional connectivity, and a network index is generated based on the brain wave data (S220). Next, whether a subject has a major depressive disorder is determined based on at least one feature data by a classification model trained to output whether a subject has a major depressive disorder with the feature data as an input (S230). Finally, a final result is provided (S240).

To be more specific, in the step S210 of receiving brain wave data of a subject, brain wave data acquired in a resting state may be acquired. At this time, the subject may be a subject which does not have a history of drug use.

Referring to FIGS. 3A and 3B together, in the step S210 of receiving brain wave data of a subject according to the exemplary embodiment of the present invention, brain wave data in a resting stage measured by a first electrode channel set configured by 62 channels of FP1, FPZ, FP2, AF3, AF4, F7, F5, F3, F1, FZ, F2, F4, F6, F8, FT7, FC5, FC3, FC1, FCZ, FC2, FC4, FC6, FT8, T7, C5, C3, C1, CZ, C2, C4, C6, T8, TP7, CP5, CP3, CP1, CPZ, CP2, CP4, CP6, TP8, P7, P5, P3, P1, PZ, P2, P4, P6, P8, PO7, PO5, PO3, POZ, PO4, PO6, PO8, CB1, O1, OZ, 02, and CB2, a second electrode channel set configured by 30 electrodes of FP1, FP2, F7, F3, FZ, F4, F8, FT7, FC3, FCZ, FC4, FT8, T7, C3, CZ, C4, T8, TP7, CP3, CPZ, CP4, TP8, P7, P3, PZ, P4, P8, O1, OZ, and O2, a third electrode channel configured by 19 electrode channels of FP1, FP2, F7, F3, FZ, F4, F8, T7, C3, CZ, C4, T8, P7, P3, PZ, P4, P8, O1, and O2, or a fourth electrode channel set configured by 10 electrode channels of F3, F4, T7, C3, C4, T8, P3, P4, O1, and O2 of the brain wave measuring device 400 may be acquired.

In the meantime, referring to FIG. 3B, when the brain wave data 410 acquired in the step S210 of receiving brain wave data of a subject includes a noise wave, the noise wave may be removed. As a result, the brain wave data 412 from which the noise wave is removed may be acquired.

According to the feature of the present invention, after the step S210 of receiving brain wave data of a subject, brain activity data may be generated based on the brain wave data.

Referring to FIG. 3B again, brain activity data 520 may be generated from the brain wave data 412 from which a noise wave is removed.

According to the feature of the present invention, in the step of generating brain activity data, the brain wave data may be converted into brain activity data by at least one of low-resolution brain electromagnetic tomography (LORETA), standardized low-resolution brain electromagnetic tomography (sLORETA), exact resolution brain electromagnetic tomography (eLORETA), minimum-norm estimate (MNE), weighted MNE (wMNE), dynamic statistical parametric mapping (dSPM), linearly constrained minimum variance (LCMV) beamformers Programs-LORETA/sLORETA toolbox, brainstrom, eConnectome, fieldtrip, and EEGlab.

According to still another feature of the present invention, after the step S210 of receiving brain wave data of a subject, filtering on the generated brain wave data may be performed. That is, brain wave data for a specific frequency may be acquired by the filtering.

For example, when the brain wave data is acquired, brain wave data in a specific frequency region, specifically, brain wave data of a delta wave δ of 1 to 4 Hz, a theta wave θ of 4 to 8 Hz, low-alpha of 8 to 10 Hz, high-alpha of 10 to 12 Hz, low-beta of 12 to 22 Hz, high-beta of 22 to 30 Hz, or a gamma wave γ of 30 to 35 Hz may be acquired. However, it is not limited thereto and the filtering on brain activity data may be performed.

In the meantime, the steps of acquiring and filtering brain wave data may be simultaneously performed. For example, when brain wave data is acquired from brain wave measurement equipment with a band pass filter, brain wave data in a specific frequency region may be acquired.

Referring to FIG. 2 again, in the step S220 of extracting a feature from the brain wave data, network structural features of the brain activity data may be determined.

According to the feature of the present invention, in the step s220 of extracting a feature from brain wave data, at least one feature of power spectrum densities (PSDs), a functional connectivity, and a network index may be extracted from the brain wave data.

According to another feature of the present invention, in the step S220 of extracting a feature from brain wave data, a functional connectivity between brain wave data may be determined.

Desirably, in the step S220 of extracting a feature from the brain wave data, a functional connectivity of PLV for the brain wave data may be determined.

According to another feature of the present invention, in the step S220 of extracting a feature from brain wave data, a functional connectivity and a network index of brain wave data may be determined. At this time, the network index may be determined based on the functional connectivity.

For example, referring to FIG. 3C together, in the step S220 of extracting a feature from brain wave data, feature data of PLV is determined from the brain wave data and a connectivity for PLV of each frequency region (δ, θ, α, β) is determined. Next, the network index is determined based on the PLV. To be more specific, a strength corresponding to a total wiring cost of the connectivity of PLV is calculated, and/or a clustering coefficient corresponding to a cluster tendency for the connectivity of PLV is calculated, and/or a path length corresponding to a length of each node in the network is determined.

According to still another feature of the present invention, in the step S220 of extracting a feature from brain wave data, the feature data may be extracted from brain wave data corresponding to a first frequency band set of theta, low-alpha, and high-alpha, a second frequency band set of delta and theta, a third frequency band set of delta, theta, low-alpha, high-alpha, and low-beta, or a fourth frequency band set of theta and low-beta.

According to still another feature of the present invention, in the step S220 of extracting a feature from brain wave data, PLV of a frequency region corresponding to a first frequency band set of theta, low-alpha, and high-alpha may be determined for the brain wave data acquired from the first electrode channel set of FP1, FPZ, FP2, AF3, AF4, F7, F5, F3, F1, FZ, F2, F4, F6, F8, FT7, FC5, FC3, FC1, FCZ, FC2, FC4, FC6, FT8, T7, C5, C3, C1, CZ, C2, C4, C6, T8, TP7, CP5, CP3, CP1, CPZ, CP2, CP4, CP6, TP8, P7, P5, P3, P1, PZ, P2, P4, P6, P8, PO7, PO5, PO3, POZ, PO4, PO6, PO8, CB1, O1, OZ, O2, and CB2.

According to still another feature of the present invention, in the step S220 of extracting a feature from brain wave data, PLV of a frequency region corresponding to a second frequency band set of delta and theta may be determined for the brain wave data acquired from the second electrode channel set of FP1, FP2, F7, F3, FZ, F4, F8, FT7, FC3, FCZ, FC4, FT8, T7, C3, CZ, C4, T8, TP7, CP3, CPZ, CP4, TP8, P7, P3, PZ, P4, P8, O1, OZ, and O2.

According to still another feature of the present invention, in the step S220 of extracting a feature from brain wave data, PLV of a frequency region corresponding to a third frequency band set of delta, theta, low-alpha, high-alpha, and low-beta may be determined for the brain wave data acquired from the third electrode channel set of FP1, FP2, F7, F3, FZ, F4, F8, T7, C3, CZ, C4, T8, P7, P3, PZ, P4, P8, O1, and O2.

According to still another feature of the present invention, in the step S220 of extracting a feature from brain wave data, PLV of a frequency region corresponding to a fourth frequency band set of theta and low-beta may be determined for the brain wave data acquired from the fourth electrode channel set of F3, F4, T7, C3, C4, T8, P3, P4, O1, and O2.

However, the combination of the number of electrode channels and the frequency band is not limited to those described above, but the feature data may be extracted from various combinations of brain wave data. Further, the feature data for the brain activity data may also be extracted.

According to still another feature of the present invention, after the step S220 of extracting a feature from the brain wave data, key features having a high level of contribution to classify the major depressive disorder or normal may be determined.

At this time, the key features may be determined based on a statistical score of a plurality of features acquired by a result of the step S220 of extracting a feature from the brain wave data.

For example, an independent sample t-test is performed on the functional connectivity, specifically, PLV, of the brain wave data determined in the step S220 of extracting a feature from the brain wave data to determine features with significant differences depending on whether a subject has the major depressive disorder. Next, after calculating a Fisher's score again on features with significant differences, key features having a level of contribution to classify the major depressive disorder may be selected by scoring the features. In the meantime, the determination of the key features is not limited to those described above but may be performed by various statistical scoring methods.

Referring to FIG. 2 again, in the step S230 of determining whether a subject has a major depressive disorder, whether the subject has the depressive disorder is determined based on a feature extracted from the brain wave data and/or brain activity data by the classification model.

For example, referring to FIG. 3B together, the feature data 522 and 524 extracted from the brain wave data and/or brain activity data is input to the classification model 530. Next, whether the major depressive order has occurred 532 is output from the classification model 530.

At this time, the classification model may be a model trained to output whether the major depressive disorder has occurred in a subject with a key feature having a high level of contribution to classify the major depressive disorder or normal as an input.

Accordingly, the classification model of the present invention may solve the problem of overfitting which is caused in the model when all feature parameters for the brain wave data are used and classify the major depressive disorder with a high accuracy.

According to the feature of the present invention, in the step S230 of determining whether the subject has the major depressive disorder, whether the subject has the major depressive disorder may be determined according to an output result of the classification model which is further configured to output 0 or 1 depending on whether the subject has the major depressive disorder.

For example, when in the step S230 of determining whether the subject has the major depressive disorder, the onset risk level of major depressive disorder of the subject is high based on the feature data, the classification model may output 1 and when a probability of normal that the onset risk level is low is high, may output 0.

Therefore, the user or the medical staff may confirm whether the major depressive disorder has occurred according to the output result (0 or 1).

As a result of the step S230 of determining whether the subject has the major depressive disorder, information associated with the major depressive disorder of the subject may be determined.

Referring to FIG. 2 again, finally, in the step S240 of providing a result, various information determined by the classification model may be output or transmitted to the user's mobile device, the medical staff device, or the like.

In the meantime, according to still another feature of the present invention, when the risk of onset of the major depressive disorder of the subject is determined, steps of receiving brain wave data, determining feature data, and re-determining whether the subject has the major depressive disorder may be repeated according to the progress of treatment.

As described above, the method for providing information on a major depressive disorder according to various exemplary embodiments of the present invention may allow a user to easily acquire information on a mental health of the user without having temporal and space restrictions. Further, medical staff may acquire information about subjects to consistently monitor the subjects suspected of suffering from a major depressive disorder, such as evaluation of a treatment prognosis.

### First Evaluation: Extract Feature for Classification of Major Depressive Disorder and Evaluate Classification Model Using the Same

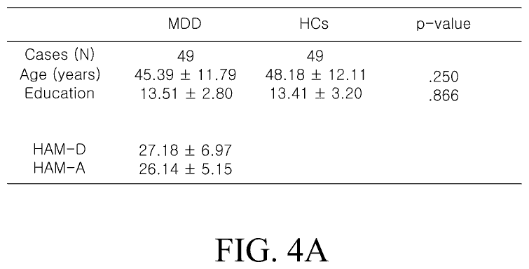

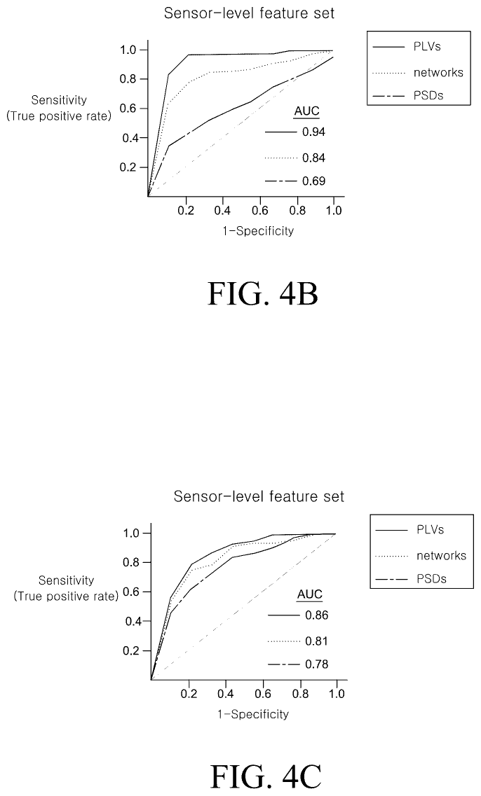

Hereinafter, an evaluation result according to a data type of a device for providing information on a major depressive disorder according to an exemplary embodiment of the present invention will be described with reference to FIGS. 4A to 4C. FIGS. 4A to 4C illustrate an evaluation result according to a feature data type of a classification model which is applied to a device for providing information on a major depressive disorder, according to an exemplary embodiment of the present invention.

First, referring to FIG. 4A, in this evaluation, brain wave data of a total of 49 subjects with a major depressive disorder (MDD) and 49 healthy controls (HC) were used. At this time, all evaluation targets are female and the subject with a major depressive disorder may be a female subject without having a history of drug use who suffered from a major depressive disorder.

To be more specific, in this evaluation, feature data of power spectrum densities (PSDs), PLV of a functional connectivity, and a network index were determined for each of the brain wave data (sensor-level data) and brain activity data (source-level data). At this time, the network index was set as a strength for the network, a clustering coefficient, and a path length. Next, the classification model classified whether all the subjects had the major depressive disorder (0 or 1) using feature data extracted from the brain wave data and feature data extracted from the brain activity data and an AUC value for the classification result was evaluated.

Referring to FIG. 4B, when the classification model based on feature data extracted from the brain wave data (sensor-level feature set) classified whether the major depressive disorder occurred using PLV of the functional connectivity, the highest AUC value of 0.94 was obtained. Next, when whether the major depressive disorder occurred was classified using the network index (strength, path length, and clustering coefficient), the AUC value was 0.84 and when whether the major depressive disorder occurred was classified using PSD, the AUC value was 0.69.

Referring to FIG. 4C together, when the classification model based on feature data extracted from the brain activity data (source-level feature set) classified whether the major depressive disorder occurred using PLV of the functional connectivity, the highest AUC value of 0.84 was obtained. Next, when whether the major depressive disorder occurred was classified using the network index (strength, path length, and clustering coefficient), the AUC value was 0.81 and when whether the major depressive disorder occurred was classified using PSD, the AUC value was 0.78.

This result may mean that whether the major depressive disorder occurs is classified based on the functional connectivity of the PLV, among feature data, specifically, PLV acquired from the brain wave data, the performance of the classification model is excellent.

Therefore, the feature data may be a functional connectivity, specifically, PLV acquired from the brain wave data, but is not limited thereto.

Therefore, the present invention may contribute to early diagnosis and good treatment prognosis of major depressive disorder by providing information on whether major depressive disorder has occurred using the classification model with excellent diagnosis performance.

### Second Evaluation: Determine Electrode Channel and Frequency Band for Classifying Major Depressive Disorder and Evaluate Classification Model Using the Same

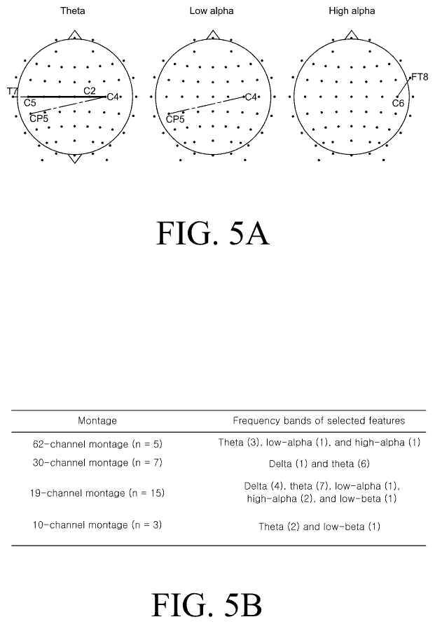

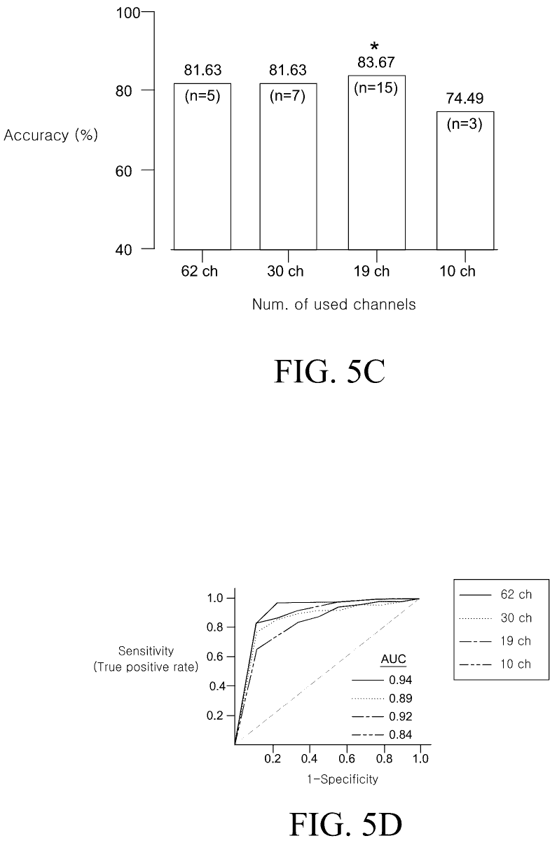

Hereinafter, an evaluation result according to a data type of a device for providing information on a major depressive disorder according to an exemplary embodiment of the present invention will be described with reference to FIGS. 5A to 5D. FIGS. 5A to 5D illustrate an evaluation result according to a brain wave data type of a classification model which is applied to a device for providing information on a major depressive disorder, according to an exemplary embodiment of the present invention.

In this evaluation, classification accuracy and an AUC value in accordance with the number of electrode channels or the reduction of the number of electrode channels were evaluated based on a PLV feature extracted from brain wave data which showed excellent classification performance in the above-described first evaluation.

First, Referring to FIG. 5A, in this evaluation, the PLV was used for each brain wave data in a resting stage measured from a first electrode channel set configured by 62 channels of FP1, FPZ, FP2, AF3, AF4, F7, F5, F3, F1, FZ, F2, F4, F6, F8, FT7, FC5, FC3, FC1, FCZ, FC2, FC4, FC6, FT8, T7, C5, C3, C1, CZ, C2, C4, C6, T8, TP7, CP5, CP3, CP1, CPZ, CP2, CP4, CP6, TP8, P7, P5, P3, P1, PZ, P2, P4, P6, P8, PO7, PO5, PO3, POZ, PO4, PO6, PO8, CB1, O1, OZ, O2, and CB2, second electrode channel set configured by 30 electrodes of FP1, FP2, F7, F3, FZ, F4, F8, FT7, FC3, FCZ, FC4, FT8, T7, C3, CZ, C4, T8, TP7, CP3, CPZ, CP4, TP8, P7, P3, PZ, P4, P8, O1, OZ, and O2, a third electrode channel set configured by 19 electrode channels of FP1, FP2, F7, F3, FZ, F4, F8, T7, C3, CZ, C4, T8, P7, P3, PZ, P4, P8, O1, and O2, or a fourth electrode channel set configured by 10 electrode channels of F3, F4, T7, C3, C4, T8, P3, P4, O1, and O2.

At this time, referring to FIG. 5B together, the brain wave data (or feature data) for each electrode channel may be data corresponding to a frequency region of theta, low-alpha, or high-alpha.

To be more specific, referring to FIG. 5A again, the PLV for the first electrode channel set may have the highest classification accuracy in a frequency region of the first frequency band set of theta, low-alpha, and high-alpha. To be more specific, the PLV for the first electrode channel set may include PLVs corresponding to three theta frequency bands, one low-alpha frequency band, and one high-alpha frequency band.

Moreover, the PLV for the second electrode channel set may have the highest classification accuracy in the frequency region of the second frequency band set of delta and theta. To be more specific, the PLV for the second electrode channel set may include PLVs corresponding to one delta frequency band and six theta frequency bands.

Moreover, the PLV for the third electrode channel set may have the highest classification accuracy in the frequency region of the third frequency band set of delta, theta, low-alpha, high-alpha, and low-beta. To be more specific, the PLV for the third electrode channel set may include PLVs corresponding to four delta frequency bands, seven theta frequency bands, one low-alpha frequency band, two high-alpha frequency bands, and one low-beta frequency band.

Moreover, the PLV for the fourth electrode channel set may have the highest classification accuracy in the frequency region of the fourth frequency band set of theta and low-beta. To be more specific, the PLV for the fourth electrode channel set may include PLVs corresponding to two theta frequency bands and one low-beta frequency band.

That is, when the major depressive disorder is classified using PLV acquired from the sensor data, regardless of the number of channels, the importance of the theta frequency may be high.

At this time, a combination of the number of electrode channels and frequency bands which are used for training the classification model to classify the major depressive disorder is not limited thereto.

Next, referring to FIG. 5C, an evaluation result of a classification model trained respectively to classify the major depressive disorder based on the PLV corresponding to the number of electrode channels and the frequency band corresponding thereto is illustrated.

To be more specific, when the classification model uses a PLV corresponding to the third frequency band set extracted from 19 channels, that is, the third electrode channel set (19 ch), the accuracy of classifying the major depressive disorder was the highest (accuracy: 83.67). Next, when a PLV based on 62 channels and 30 channels, that is, the first electrode channel set and the second electrode channel set (62 ch and 30 ch) was used, the accuracy of classifying the major depressive disorder was high (each accuracy: 81.63).

That is, this result may mean that even though the number of electrode channels was reduced from 62 or 30 to 19, the accuracy of classifying the major depressive disorder was not lowered but was increased.

Referring to FIG. 5D together, the AUC value of the classification model based on the third electrode channel set with 19 electrode channels was 0.92 which was similar to the AUC value of 0.94 based on the first electrode channel set with 62 electrode channels.

That is, this result may mean that even though the number of electrode channels was reduced from 62 or 30 to 19, the classification performance of the major depressive disorder of the classification model was maintained.

Therefore, the classification model used for various exemplary embodiments of the present invention may be a model trained to output whether a major depressive disorder occurs with a PLV acquired from the third electrode channel set including 19 electrode channels of FP1, FP2, F7, F3, FZ, F4, F8, T7, C3, CZ, C4, T8, P7, P3, PZ, P4, P8, O1, and O2, specifically, the PLV corresponding to the frequency region of the third frequency band set of delta, theta, low-alpha, high-alpha, and low-beta as an input. Specifically, the classification model may be a model trained to classify whether a major depressive disorder occurs in female subjects without a history of drug use. However, data used for training a classification model and actual classification is not limited to those described above.

Although the exemplary embodiments of the present invention have been described in detail with reference to the accompanying drawings, the present invention is not limited thereto and may be embodied in many different forms without departing from the technical concept of the present invention. Therefore, the exemplary embodiments of the present invention are provided for illustrative purposes only but are not intended to limit the technical concept of the present invention. The scope of the technical concept of the present invention is not limited thereto. Therefore, it should be understood that the above-described exemplary embodiments are illustrative in all aspects and do not limit the present invention. The protective scope of the present invention should be construed based on the following claims, and all the technical concepts in the equivalent scope thereof should be construed as falling within the scope of the present invention.

1. A method for providing information on a major depressive disorder being implemented by a processor, comprising:
receiving brain wave data of a subject;
extracting at least one feature data of power spectrum densities (PSDs), a functional connectivity, and a network index with respect to the brain wave data; and
determining whether the subject has the major depressive disorder on the basis of the at least one feature data, by using a classification model trained to output whether the subject has the major depressive disorder on the basis of the at least one feature data as an input,
wherein the subject is a subject suspected of suffering from the major depressive disorder without having a history of drug use.

2. The method of claim 1, wherein:
the receiving brain wave data includes:
receiving brain wave data of the subject measured from a plurality of electrode channels selected from FP1, FPZ, FP2, AF3, AF4, F7, F5, F3, F1, FZ, F2, F4, F6, F8, FT7, FC5, FC3, FC1, FCZ, FC2, FC4, FC6, FT8, T7, C5, C3, C1, CZ, C2, C4, C6, T8, TP7, CP5, CP3, CP1, CPZ, CP2, CP4, CP6, TP8, P7, P5, P3, P1, PZ, P2, P4, P6, P8, PO7, PO5, PO3, POZ, PO4, PO6, PO8, CB1, O1, OZ, O2, and CB2 and
extracting at least one feature data includes:
extracting the at least one feature data from a plurality of frequency bands selected from low-alpha, high-alpha, low-beta, high-beta, gamma, delta, and theta of the brain wave data measured from the plurality of electrode channels.

3. The method of claim 2, wherein:
the plurality of electrode channels is a first electrode channel set of FP1, FPZ, FP2, AF3, AF4, F7, F5, F3, F1, FZ, F2, F4, F6, F8, FT7, FC5, FC3, FC1, FCZ, FC2, FC4, FC6, FT8, T7, C5, C3, C1, CZ, C2, C4, C6, T8, TP7, CP5, CP3, CP1, CPZ, CP2, CP4, CP6, TP8, P7, P5, P3, P1, PZ, P2, P4, P6, P8, PO7, PO5, PO3, POZ, PO4, PO6, PO8, CB1, O1, OZ, O2, and CB2, a second electrode channel set of FP1, FP2, F7, F3, FZ, F4, F8, FT7, FC3, FCZ, FC4, FT8, T7, C3, CZ, C4, T8, TP7, CP3, CPZ, CP4, TP8, P7, P3, PZ, P4, P8, O1, OZ, and O2, a third electrode channel set of FP1, FP2, F7, F3, FZ, F4, F8, T7, C3, CZ, C4, T8, P7, P3, PZ, P4, P8, O1, and O2, or a fourth electrode channel set of F3, F4, T7, C3, C4, T8, P3, P4, O1, and O2 and
the plurality of frequency bands is a first frequency band set of theta, low-alpha, and high-alpha, a second frequency band set of delta and theta, a third frequency band set of delta, theta, low-alpha, high-alpha, and low-beta, or a fourth frequency band set of theta and low-beta.

4. The method of claim 3, wherein:
the plurality of electrode channels is the first electrode channel set, and the plurality of frequency bands is the first frequency band set.

5. The method of claim 3, wherein:
the plurality of electrode channels is the second electrode channel set, and the plurality of frequency bands is the second frequency band set.

6. The method of claim 3, wherein:
the plurality of electrode channels is the third electrode channel set, and the plurality of frequency bands is the third frequency band set.

7. The method of claim 3, wherein:
the plurality of electrode channels is the fourth electrode channel set, and the plurality of frequency bands is the fourth frequency band set.

8. The method of claim 1, wherein the at least one feature data is the functional connectivity and extracting at least one feature data includes:
determining a connectivity of a phase locking value (PLV) for the brain wave data.

9. The method of claim 1, wherein:
the at least one feature data is the functional connectivity and the network index and
extracting at least one feature data includes:
extracting the functional connectivity from the brain wave data; and
determining the network index based on network structural feature data of the functional connectivity.

10. The method of claim 9, wherein determining the network index includes:
determining at least one index of a strength for the functional connectivity, a clustering coefficient, and a path length.

11. The method of claim 1, further comprising:
generating brain activity data based on the brain wave data, after the receiving brain wave data
wherein:
extracting at least one feature data further includes:
extracting the at least one feature data for each of the brain wave data and the brain activity data, and
the determining of whether the subject has a major depressive disorder further includes:
determining whether the subject has a major depressive disorder using the classification model, based on the feature data for each of the brain wave data and the brain activity data.

12. The method of claim 1, further comprising:
filtering the brain activity data based on a band pass filter, which is performed after generating the brain activity data.

13. The method of claim 1, wherein the brain wave data is defined as brain wave data acquired in a resting state.

14. A device for providing information on a major depressive disorder, comprising:
a communication unit configured to receive brain wave data of a subject; and
a processor connected to communicate with the communication unit,
wherein the processor is configured to extract at least one feature data of power spectrum densities (PSDs), a functional connectivity, and a network index with respect to the brain wave data and determine whether the subject has the major depressive disorder on the basis of the at least one feature data, by using a classification model trained to output whether the subject has the major depressive disorder on the basis of the at least one feature data as an input, and
the subject is a subject suspected of suffering from the major depressive disorder without having a history of drug use.

15. The device of claim 14, wherein:
the communication unit is configured to receive the brain wave data of the subject measured from a plurality of electrode channels selected from FP1, FPZ, FP2, AF3, AF4, F7, F5, F3, F1, FZ, F2, F4, F6, F8, FT7, FC5, FC3, FC1, FCZ, FC2, FC4, FC6, FT8, T7, C5, C3, C1, CZ, C2, C4, C6, T8, TP7, CP5, CP3, CP1, CPZ, CP2, CP4, CP6, TP8, P7, P5, P3, P1, PZ, P2, P4, P6, P8, PO7, PO5, PO3, POZ, PO4, PO6, PO8, CB1, O1, OZ, O2, and CB2 and
the processor is configured to extract the at least one feature data from a plurality of frequency bands selected from low-alpha, high-alpha, low-beta, high-beta, gamma, delta, and theta of the brain wave data measured from the plurality of electrode channels.

16. The device of claim 15, wherein:
the plurality of electrode channels is a first electrode channel set of FP1, FPZ, FP2, AF3, AF4, F7, F5, F3, F1, FZ, F2, F4, F6, F8, FT7, FC5, FC3, FC1, FCZ, FC2, FC4, FC6, FT8, T7, C5, C3, C1, CZ, C2, C4, C6, T8, TP7, CP5, CP3, CP1, CPZ, CP2, CP4, CP6, TP8, P7, P5, P3, P1, PZ, P2, P4, P6, P8, PO7, PO5, PO3, POZ, PO4, PO6, PO8, CB1, O1, OZ, O2, and CB2, a second electrode channel set of FP1, FP2, F7, F3, FZ, F4, F8, FT7, FC3, FCZ, FC4, FT8, T7, C3, CZ, C4, T8, TP7, CP3, CPZ, CP4, TP8, P7, P3, PZ, P4, P8, O1, OZ, and O2, a third electrode channel set of FP1, FP2, F7, F3, FZ, F4, F8, T7, C3, CZ, C4, T8, P7, P3, PZ, P4, P8, O1, and O2, or a fourth electrode channel set of F3, F4, T7, C3, C4, T8, P3, P4, O1, and O2 and
the plurality of frequency bands is a first frequency band set of theta, low-alpha, and high-alpha, a second frequency band set of delta and theta, a third frequency band set of delta, theta, low-alpha, high-alpha, and low-beta, or a fourth frequency band set of theta and low-beta.

17. The device of claim 16, wherein the plurality of electrode channels is the first electrode channel set, and the plurality of frequency bands is the first frequency band set.

18. The device of claim 16, wherein the plurality of electrode channels is the second electrode channel set, and the plurality of frequency bands is the second frequency band set.

19. The device of claim 16, wherein the plurality of electrode channels is the third electrode channel set, and the plurality of frequency bands is the third frequency band set.

20. The device of claim 16, wherein the plurality of electrode channels is the fourth electrode channel set, and the plurality of frequency bands is the fourth frequency band set.

21. (canceled)

22. (canceled)

23. (canceled)

24. (canceled)
